(19) United States
(12) Patent Application Publication
(10) Pub. No.: US 2018/0374211 A1
(43) Pub. Date: Dec. 27, 2018

(54) INFORMATION PROCESSING APPARATUS, AND PROGRAM, METHOD AND SYSTEM THEREOF

(71) Applicants: TAKANO CO., LTD., Nagano (JP); NATIONAL UNIVERSITY CORPORATION CHIBA UNIVERSITY, Chiba-shi (JP)

(72) Inventors: Tomohiro KURATA, Nagano (JP); Hideaki HANEISHI, Chiba (JP)

(21) Appl. No.: 15/869,140

(22) Filed: Jan. 12, 2018

(30) Foreign Application Priority Data
Jun. 26, 2017 (JP) 2017-124176

(51) Int. Cl. G06T 7/00; A61B 5/1455; A61B 5/00; G06T 7/70

(52) U.S. Cl. CPC G06T 7/0012; A61B 5/14551; G06T 2207/30101; A61B 5/0082; G06T 7/70; A61B 5/0064

(57) ABSTRACT

An information processing apparatus includes: a memory that stores a predetermined instruction command, and stores an image showing a blood vessel of a biological tissue imaged by a probe; and a processor configured to execute the instruction command stored in the memory, to generate an index indicating the state of blood in the blood vessel at one or more coordinate positions in the image, and output each generated index associated with each corresponding coordinate position.

### BACKGROUND

### Technical Field

The present disclosure relates to an information processing apparatus for analyzing the state of a biological tissue, a program to be executed and a method to be implemented by the information processing apparatus, and a system using the information processing apparatus.

### Related Art

There have been apparatuses that emit light onto a biological tissue and detect light reflected from the biological tissue, to analyze the state of the biological tissue. For example, JP 2003-220033 A discloses an apparatus that emits excitation light onto a biological tissue from a probe to which an excitation light source is connected, and detects the intensity of fluorescence emitted from the biological tissue excited by the excitation light.

### SUMMARY

In view of the above technologies, the present disclosure provides an information processing apparatus, a program, a method, and a system for analyzing the state of blood in a biological tissue, particularly blood in a blood vessel.

An aspect of the present disclosure provides “an information processing apparatus that includes: a memory configured to store a predetermined instruction command, and store an image showing a blood vessel of a biological tissue imaged by a probe; and a processor configured to execute the instruction command stored in the memory, to generate an index indicating a state of blood in the blood vessel at one or a plurality of coordinate positions in the image, and output each generated index associated with each corresponding coordinate position”.

An aspect of the present disclosure provides “a non-transitory computer-readable storage medium storing a program to be executed by a computer including a memory storing an image showing a blood vessel of a biological tissue imaged by a probe, the program being for causing the computer to function as a processor configured to execute processing to generate an index indicating a state of blood in the blood vessel at one or a plurality of coordinate positions in the image and output each generated index associated with each corresponding coordinate position”.

An aspect of the present disclosure provides “a method implemented by a processor executing a predetermined instruction command stored in a memory, the method including: storing an image showing a blood vessel of a biological tissue imaged by a probe; generating an index indicating a state of blood in the blood vessel at one or a plurality of coordinate positions in the image; and outputting each generated index associated with each corresponding coordinate position”.

An aspect of the present disclosure provides “a system that includes: an information processing apparatus and a probe, the probe including: a light source that is capable of emitting a plurality of light beams having different peak wavelength regions, the light source being communicably connected to the information processing apparatus; and an image sensor that detects light reflected from a surface of a biological tissue among the light beams emitted from the light source”.

According to various embodiments of the present disclosure, it is possible to provide an information processing apparatus, a program, a method, and a system for analyzing the state of blood in a biological tissue, particularly blood in a blood vessel.

It should be noted that the above mentioned effect is merely an example for ease of explanation, and does not limit the scope of the invention. In addition to or in place of the above effect, it is also possible to achieve any of the effects described in the present disclosure and effects obvious to those skilled in the art.

### BRIEF DESCRIPTION OF DRAWINGS

FIG. 1 is a diagram for explaining the configuration of a system 1 according to an embodiment of the present disclosure;

FIG. 2 is a block diagram showing example configurations of an information processing apparatus 100, a probe 200, and a light source control device 300 that constitute the system 1 according to the embodiment of the present disclosure;

FIG. 3A is a conceptual diagram showing a cross-section of the structure of the probe 200 according to the embodiment of the present disclosure;

FIG. 3B is a conceptual diagram showing a bottom surface of the structure of the probe 200 according to the embodiment of the present disclosure;

FIG. 4 is a conceptual diagram showing a utility form of the probe 200 according to the embodiment of the present disclosure;

FIG. 5 is a diagram showing the flow in a process to be performed in the system 1 according to the embodiment of the present disclosure;

FIG. 6 is a diagram showing the flow in a process to be performed in the information processing apparatus 100 according to the embodiment of the present disclosure;

FIG. 7A is a diagram showing an example of an image captured via the probe 200 according to the embodiment of the present disclosure;

FIG. 7B is a diagram showing an example of an image processed in the information processing apparatus 100 according to the embodiment of the present disclosure;

FIG. 7C is a diagram showing an example of an image processed in the information processing apparatus 100 according to the embodiment of the present disclosure;

FIG. 7D is a diagram showing an example of an image processed in the information processing apparatus 100 according to the embodiment of the present disclosure;

FIG. 7E is a diagram showing an example of an image processed in the information processing apparatus 100 according to the embodiment of the present disclosure;

FIG. 7F is a diagram showing an example of an image processed in the information processing apparatus 100 according to the embodiment of the present disclosure;

FIG. 8 is a diagram showing the flow in a process to be performed in the information processing apparatus 100 according to the embodiment of the present disclosure;

FIG. 9 is a diagram showing an example of an image outputted from the information processing apparatus 100 according to the embodiment of the present disclosure; and

FIG. 10 is a diagram showing an example of an image outputted from the information processing apparatus 100 according to the embodiment of the present disclosure.

### DETAILED DESCRIPTION

The following is a description of various embodiments of the present disclosure, with reference to the accompanying drawings. It should be noted that, in the drawings, like components are denoted by like reference numerals.

### 1. Overview of a System According to the Present Disclosure

One of the example systems according to various embodiments of the present disclosure is a system that captures an image of a microcirculating system (blood vessels such as the arterioles, the capillaries, or the venules, for example) of a biological tissue (an organ, for example) with a probe, generates indices indicating the states of blood (such as the oxygen saturation level of the blood) in the blood vessels at one or more coordinate positions in the captured image showing the blood vessels, associates the generated indices with the respective coordinate positions, and outputs the associated indices and coordinate positions.

A specific example of such a system captures an image of a capillary vessel in the surface of a human biological tissue with a probe. The captured image is transferred from the probe to an information processing apparatus. The information processing apparatus performs various kinds of image processing and image analysis, and estimates the oxygen saturation level of the blood at one or more coordinate positions in the image. The indices generated through the estimation of the oxygen saturation level are arranged (mapped) in an overlapping manner at the coordinate positions in the captured image, and the resultant image is displayed on a display or the like of the information processing apparatus.

An index indicating the state of the blood in a blood vessel may be any kind of index that can be acquired from an image captured with a probe. However, preferred examples include the oxygen saturation level of the blood, the total hemoglobin concentration in the blood, and a combination thereof.

Further, an index indicating the state of the blood in a blood vessel may be the numerical value of a calculated or estimated oxygen saturation level or the total concentration. Alternatively, such numerical values may be classified into predetermined ranges. That is, the index is not necessarily the numerical value of a calculated or estimated oxygen saturation level or the total concentration, but may be information processed in accordance with the numerical value.

Further, when an image is outputted to a display or the like, the image in which generated indices are mapped may be an image captured with a probe, or may be an image subjected to image processing such as smoothing, binarization, or normalization.

### 2. Configuration of a System 1 According to an Embodiment

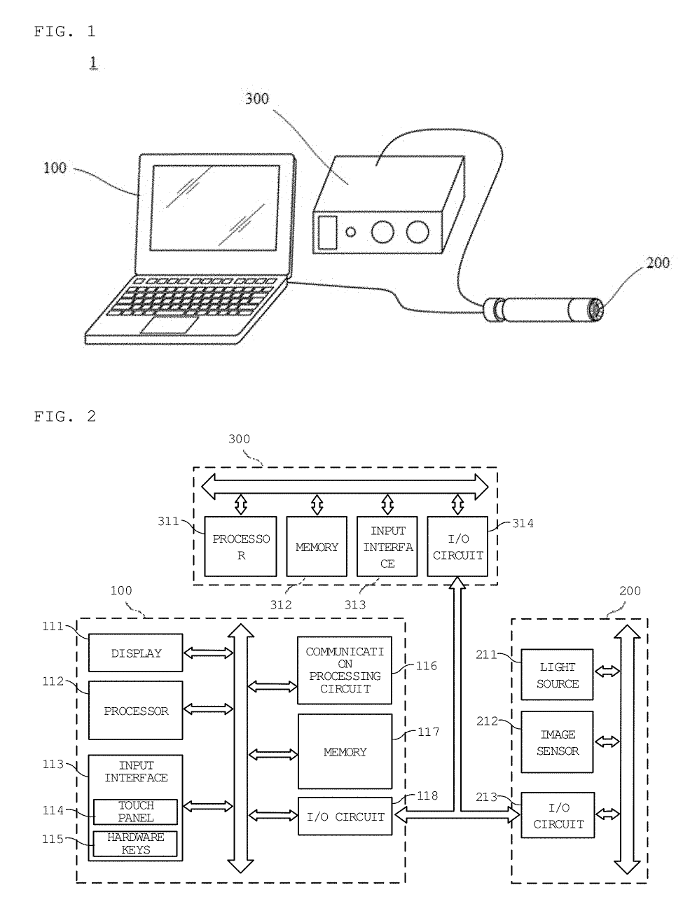

FIG. 1 is a diagram for explaining a system according to an embodiment of the present disclosure. Referring to FIG. 1, the system 1 includes: a probe 200 for capturing an image of a biological tissue; an information processing apparatus 100 that performs processing and the like on the captured image; a light source control device 300 that controls a light source included in the probe 200. The probe 200, the information processing apparatus 100, and the light source control device 300 are connected to one another so as to be capable of transmitting and receiving various kinds of information, instruction commands, data, and the like. Among these components, the probe 200 is used while being in contact with the surface of a biological tissue, to enable dark field imaging, instead of conventional imaging with a bright field.

Although the light source control device 300 is provided in FIG. 1, it is also possible to eliminate the light source control device 300 by controlling the light source of the probe 200 with a microprocessor or the like in the information processing apparatus 100 or the probe 200. Further, the information processing apparatus 100 is shown as a component, but it is also possible to provide an information processing apparatus for each of various processes and each kind of information to be stored.

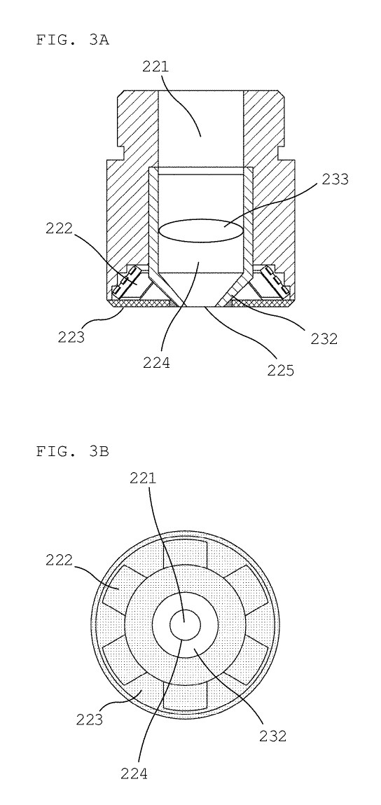

FIG. 2 is a block diagram showing example configurations of the information processing apparatus 100, the probe 200, and the light source control device 300 that constitute the system 1 according to the embodiment of the present disclosure. It should be noted that the information processing apparatus 100, the probe 200, and the light source control device 300 do not necessarily include all of the components shown in FIG. 2. Some of the components may be excluded, or some other components may be added to the components shown in FIG. 2.

Referring to FIG. 2, the information processing apparatus 100 includes a display 111, a processor 112, an input interface 113 including a touch panel 114 and hardware keys 115, a communication processing circuit 116, a memory 117, and an I/O circuit 118. These components are electrically connected to one another via a control line or a data line.

The display 111 functions as a display module that reads out image information stored in the memory 117 and performs various outputs in response to an instruction from the processor 112. Specifically, the display 111 displays an image in which an index indicating the state of the blood in a blood vessel generated by the processor 112 is mapped on an image of the blood vessel, and displays various setting screens for generating the mapping image or images of the generation process. The display 111 is formed with a liquid crystal display, for example.

The processor 112 is formed with a CPU (a microcomputer), for example, and executes an instruction command (a program) stored in the memory 117, to function as a controller for controlling the other connected components. For example, the processor 112 executes various image analysis programs stored in the memory 117, to generate indices indicating the states of blood in the blood vessels at one or more coordinate positions in an image showing the blood vessels imaged by the probe 200, arranges the generated indices at the one or more coordinate positions in the captured images, and displays the indices on the display 111. It should be noted that the processor 112 may be formed with a single CPU, or may be formed with two or more CPUs. Further, some other kind of processor such as a GPU specialized for image processing may be appropriately combined with the processor 112.

The input interface 113 includes the touch panel 114 and/or the hardware keys 115, and functions as an operation module that accepts various instructions and inputs from the user. The touch panel 114 is disposed so as to cover the display 111, and outputs information about the positional coordinates corresponding to the image data displayed on the display 111 to the processor 112. As a touch panel system, a known system such as a resistive film system, a capacitive coupling system, or an ultrasonic surface acoustic wave system can be used.

The communication processing circuit 116 performs processing such as modulation and demodulation to transmit and receive information to and from a server apparatus or another information processing apparatus installed at a remote location via a connected antenna (not shown). For example, the communication processing circuit 116 performs processing to transmit a mapping image obtained as a result of executing a program according to this embodiment to the server apparatus or another information processing apparatus. It should be noted that the communication processing circuit 116 performs processing according to a wideband wireless communication system such as the Wideband-Code Division Multiple Access (W-CDMA) system, but may also perform processing according to a narrowband wireless communication system such as a wireless LAN, typically IEEE 802.11, or Bluetooth (registered trademark). Alternatively, the communication processing circuit 116 can use known wired communications.

The memory 117 is formed with a ROM, a RAM, a nonvolatile memory, an HDD, and the like, and functions as a storage. The ROM stores instruction commands for performing image processing and the like according to this embodiment and a predetermined OS as a program. The RAM is a memory used for writing and reading data while the program stored in the ROM is being processed by the processor 112. The nonvolatile memory or the HDD is a memory in which data writing and reading is performed as the program is executed, and the data written therein is saved even after the execution of the program is completed. For example, images such as images captured by the probe 200, images such as mapping images, and information about the user who is the object of imaging being performed by the probe 200 are stored in the nonvolatile memory or the HDD.

The I/O circuit 118 is connected to the I/O circuits included in the probe 200 and the light source control device 300, and functions as an information input/output module for inputting/outputting information to/from the probe 200 and the light source control device 300. Specifically, the I/O circuit 118 functions as an interface for receiving an image captured by the probe 200 and for transmitting a control signal for controlling the image sensor 212 included in the probe 200. It should be noted that the I/O circuit 118 can adopt a known connection form, such as a serial port, a parallel port, or a USB, as desired.

Referring to FIG. 2, the probe 200 includes a light source 211, an image sensor 212, and an I/O circuit 213. These components are electrically connected to one another via a control line or a data line.

The light source 211 is formed with at least one LED. For example, the light source 211 is formed with light sources having different peak wavelengths: an LED for emitting blue light with a peak wavelength of 470 nm and a half-value width of 30 nm to a biological tissue or blood vessels, and an LED for emitting green light having a peak wavelength of 527 nm and a half-value width of 30 nm to a biological tissue or blood vessels. The luminescent color of the light source is not limited to the above particular luminescent colors, as long as the peak wavelengths of the luminescent colors fall within the range of 400 nm to 600 nm, which is a wavelength region in which the light absorption by the hemoglobin contained in the blood is dominant. Although the light source that emits the two kinds of light, blue light and green light, is described above, it is also possible to provide another light source having different peak wavelength regions. In a case where a light source (of red light, for example) having no peak wavelengths in the above light absorption wavelength region is used, the difference in light absorption between the blood vessel portion and its surrounding portion is small. In a case where a light source having a peak wavelength region in the light absorption wavelength region of hemoglobin is used, on the other hand, the difference in light absorption between the blood vessel portion and its surrounding portion is sufficiently large. In such a case, the difference in pixel value between the blood vessel portion and its surrounding portion in the image captured by the probe 200 is clearer, and thus, it is possible to extract the blood vessel portion in a preferred manner.

Although not shown, the light source 211 may include a known switching circuit for cyclically switching its luminescent colors (peak wavelengths) in accordance with a control signal received from the processor 311 of the light source control device 300.

The image sensor 212 captures an image of the imaging object by detecting light scattered in a biological tissue and reflected from the surface of the biological tissue, and generates an image signal to be outputted to the information processing apparatus 100 via the I/O circuit 213. As the image sensor 212, a known image sensor such as a charge coupled device (CCD) imaging sensor or a complementary metal-oxide semiconductor (CMOS) imaging sensor can be used. The generated image signal is processed by the respective circuits such as a CDS circuit, an AGC circuit, and an A/D converter, and is then transmitted as a digital image signal to the information processing apparatus 100.

The I/O circuit 213 is connected to the respective I/O circuits included in the information processing apparatus 100 and the light source control device 300, and functions as an information input/output module that stores information for inputting/outputting information from/to the information processing apparatus 100 and the light source control device 300. Specifically, the I/O circuit 213 functions as an interface for transmitting a digital image signal generated by the image sensor 212 or the like to the information processing apparatus 100, and receiving control signals for controlling the light source 211 and the image sensor 212 from the information processing apparatus 100 and the light source control device 300. It should be noted that the I/O circuit 213 can adopt a known connection form, such as a serial port, a parallel port, or a USB, as desired.

Referring to FIG. 2, the light source control device 300 includes a processor 311, a memory 312, an input interface 313, and an I/O circuit 314. These components are electrically connected to one another via a control line or a data line.

The processor 311 is formed with a CPU (a microcomputer), for example, and executes instruction commands (various programs, for example) stored in the memory 312, to function as a controller for controlling the other connected components. For example, the processor 311 executes a light source control program stored in the memory 312, and outputs a control signal for cyclically switching the color of light to be outputted from the light source 211 provided in the probe 200. It should be noted that the processor 311 may be formed with a single CPU, or may be formed with two or more CPUs.

The memory 312 is formed with a ROM, a RAM, a nonvolatile memory, an HDD, and the like, and functions as a storage. The ROM stores instruction commands for performing light source control according to this embodiment and a predetermined OS as a program. The RAM is a memory used for writing and reading data while the program stored in the ROM is being processed by the processor 311. The nonvolatile memory or the HDD is a memory in which data writing and reading is performed as the program is executed, and the data written therein is saved even after the execution of the program is completed. For example, the nonvolatile memory and the HDD store setting information such as the peak wavelength of the light source, the light emission cycle of light to be emitted from the light source (or the switching cycle in a case where two or more luminescent colors are used).

The input interface 313 is formed with hardware keys and the like, and functions as an operation module that accepts various kinds of setting information of the light source from the user.

The I/O circuit 314 is connected to the respective I/O circuits included in the information processing apparatus 100 and the probe 200, and functions as an information input/output module for inputting/outputting information from/to the information processing apparatus 100 and the probe 200. Specifically, the I/O circuit 314 functions as an interface for transmitting a control signal for controlling the light source 211 of the probe 200, to the probe 200. It should be noted that the I/O circuit 314 can adopt a known connection form, such as a serial port, a parallel port, or a USB, as desired.

### 3. Structure of the Probe 200

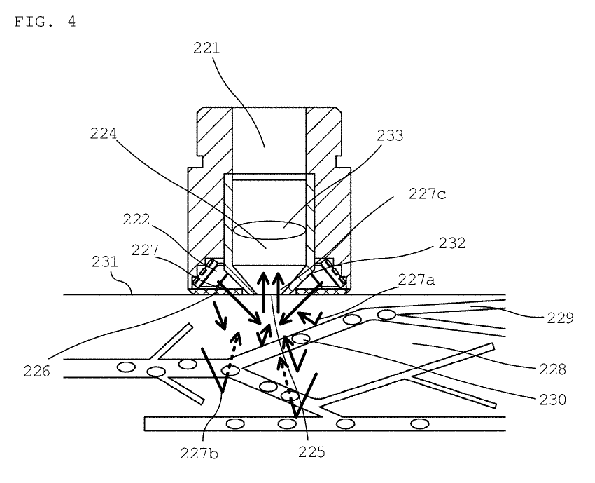

FIG. 3A is a conceptual diagram showing a cross-section of the structure of the probe 200 according to the embodiment of the present disclosure. FIG. 3B is a conceptual diagram showing a bottom surface of the structure of the probe 200 according to the embodiment of the present disclosure. As shown in FIGS. 3A and 3B, to deliver light to an image sensor 221 provided in the camera through a contact surface 225 in contact with the surface of a biological tissue, the probe 200 has an optical path 224 that is disposed between the contact surface 225 and the image sensor 221. A lens 233, an optical filter, and the like may be disposed in the optical path 224 in accordance with desired image data and the position of the image sensor 221.

The probe 200 also includes LEDs 222 as light sources disposed around the optical path 224, and a separation wall 232 that is formed around the optical path 224 and is designed to physically separate the optical path 224 from the LEDs 222. The LEDs 222 are completely separated from the optical path 224 leading to the image sensor 221 by the separation wall 232 in optical terms, to capture images of biological tissues by a dark field imaging method (specifically, a side stream dark field imaging method). Specifically, the LEDs 222 are installed so that the optical axis of the light to be emitted to a biological tissue as the object is tilted at a predetermined angle (about 50 degrees, for example) with respect to the optical axis of the light passing through the optical path 224. As light emitted from the LEDs 222 has directivity, it is possible not only to completely separate the LEDs 222 from the optical path 224 in optical terms, but also to increase the intensity of the light to be emitted to the biological tissue as the object. In the example shown in FIGS. 3A and 3B, the probe 200 includes six multicolor LEDs 222 around the optical path 224. As the LEDs 222 are arranged at even intervals in this manner, light can be uniformly emitted onto the object.

In the example shown in FIGS. 3A and 3B, multicolor LEDs are used so that light colors (blue light and green light, for example) are switched at predetermined intervals, in accordance with a control signal from the light source control device 300. An example of the switching cycle is 500 msec, or preferably 200 msec, or more preferably 100 msec.

It should be noted that the present invention is not limited to this, and it is also possible to adopt two or more kinds of light sources of different luminescent colors in advance. In the example shown in FIGS. 3A and 3B, the six LEDs 222 are used, but it is of course possible to increase or decrease the number of LEDs 222 as desired. For example, it is possible to use only one LED or use eight LEDs.

Further, the probe 200 is provided with a cover 223 on the contact surface to be brought into contact with a biological tissue, so that the LEDs 222 are covered. The cover 223 is made of a silicone resin, for example, and prevents the LEDs 222 from being brought into direct contact with a biological tissue and its secretion, and being contaminated.

FIG. 4 is a conceptual diagram showing a utility form of the probe 200 according to the embodiment of the present disclosure. In this embodiment, an image captured by the probe 200 is an image captured according to a dark field imaging method. Therefore, light emitted from the LEDs 222 needs to be optically separated from the optical path 224. In view of this, the above image is captured while the contact surface 225 and the cover surface 226 of the probe 200 are in contact with the surface 231 of a biological tissue 228.

Specifically, as shown in FIG. 4, light (blue light or green light, for example) emitted from the LEDs 222 passes through the cover surface 226 and the surface 231 of the biological tissue 228, and then enters the biological tissue 228. The incident light 227 is scattered in the biological tissue 228 like light 227a. At this stage, the incident light 227 has its peak wavelength in the absorption wavelength region of the hemoglobin of the red blood cells. Therefore, part of the scattered light 227a (light 227b, for example) is absorbed by the hemoglobin 230 of the red blood cells contained in a capillary vessel 229 in the vicinity of the surface 231. On the other hand, part of the light not absorbed by the hemoglobin 230 of the red blood cells (light 227c, for example) passes through the surface 231 of the biological tissue 228 and the contact surface 225 of the probe 200, and then enters the optical path 224. The light 227c finally reaches the image sensor 221, and is imaged by the image sensor 221.

As described above, in this embodiment, the probe 200 is used while the contact surface 225 and the cover surface 226 of the probe 200 are in contact with the surface 231 of the biological tissue 228. Thus, light reflection from the surface 231 of the biological tissue 228 can be reduced. Further, as a dark field imaging method is used, clearer imaging of the capillary vessel 229 is enabled.

### 4. Outline of a Process to Be Performed by the Information Processing Apparatus 100

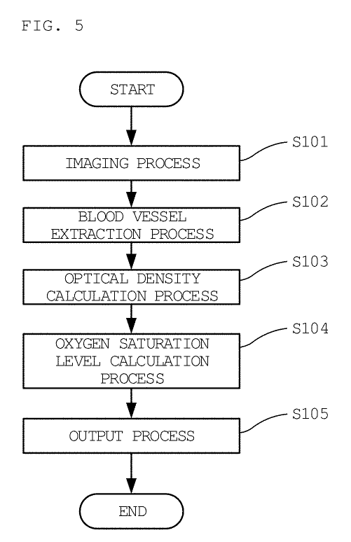

FIG. 5 is a diagram showing the flow in a process to be performed in the system 1 according to the embodiment of the present disclosure. Specifically, FIG. 5 is a diagram showing the flow in a process to be performed by the processor 112 of the information processing apparatus 100 and the processor 311 of the light source control device 300 executing instruction commands stored in the respective memories 117 and 312.

As shown in FIG. 5, the process is started when the probe 200 receives a control signal from the processor 311 as a result of setting of the LEDs 222 as the light sources in the light source control device 300, and a control signal for imaging from the processor 112 of the information processing apparatus 100. First, the probe 200 that has received the control signals controls the peak wavelength of light to be emitted from the LEDs 222 and the switching cycle thereof, and emits light to the biological tissue to be imaged. The probe 200 then detects scattered light received by the image sensor 212, and captures an image showing the blood vessels of the biological tissue (S101). At this stage, the blue light and the green light are switched at predetermined switching intervals as described above, and the blue light and the green light are separately detected in the image sensor 212. Therefore, in the imaging process, two spectral images, a spectral image of blue light and a spectral image of green light, are obtained.

In the imaging process, the depth of focus is 5.6 mm, the color switching cycle of the LEDs 222 is 100 msec, and the frame rate is 30 fps, for example. Through the imaging process, an image of 640×640 pixels is generated.

Each of the captured spectral images is transmitted to the information processing apparatus 100 via the I/O circuit 213 of the probe 200 and the I/O circuit 118 of the information processing apparatus 100. Each spectral image is stored into the memory 117 under the control of the processor 112. The processor 112 reads each spectral image and instruction commands (a program) for processing the spectral images from the memory 117, and performs a process of extracting a blood vessel region from each spectral image (S102). In the blood vessel extraction process, it is possible to combine a process of extracting a tubular structure in accordance with a Hessian matrix, a binarization process, an analysis process based on pixel values, and the like as appropriate, and perform the combined process on each spectral image, for example.

After the coordinate positions of the blood vessels shown in the image are identified through the above blood vessel extraction process, the processor 112 performs a process of calculating the optical density at one or more coordinate positions indicating the blood vessels in accordance with an instruction command stored in the memory 117 (S103). It should be noted that the optical density is calculated in accordance with the pixel value of the portion extracted as a blood vessel and the average pixel value of the background portion around the blood vessel portion, for example.

The processor 112 then performs a process of generating an index (indices) indicating the oxygen saturation levels of the blood at one or more coordinate positions in accordance with an instruction command stored in the memory 117 (S104). It should be noted that the oxygen saturation level calculation process is performed by using the calculated optical density, the molar absorption coefficients of oxygenated hemoglobin and deoxygenated hemoglobin, and the like.

After the index (indices) indicating the oxygen saturation level(s) at one or more coordinate positions corresponding to the blood vessels in the image is/are generated through the above described oxygen saturation level calculation process, the processor 112 performs a process of outputting the indices associated with the respective coordinate positions, in accordance with an instruction command stored in the memory 117 (S105). For example, in accordance with the coordinate positions, the respective indices are arranged in one of the spectral images received from the probe 200 or in a processed image created in accordance with the respective spectral images during the above processes, and the indices are then outputted to the display 111 of the information processing apparatus 100.

In the above manner, from the image captured by the probe 200, indices based on the oxygen saturation levels are generated as indices indicating the state of the blood in the blood vessel, and the series of processes till the outputting of the indices to the display 111 comes to an end. Each of these processes will be described later in detail.

### 5. Blood Vessel Extraction Process

FIG. 6 is a diagram showing the flow in a process to be performed in the information processing apparatus 100 according to the embodiment of the present disclosure. Specifically, FIG. 6 is a diagram showing the flow in a process to be performed by the processor 112 of the information processing apparatus 100 executing an instruction command stored in the memory 117.

First, the processor 112 controls the I/O circuit 118 and the memory 117 so that the I/O circuit 118 of the information processing apparatus 100 receives an image showing the blood vessels in a biological tissue imaged by the probe 200, and stores the image into the memory 117 (S201).

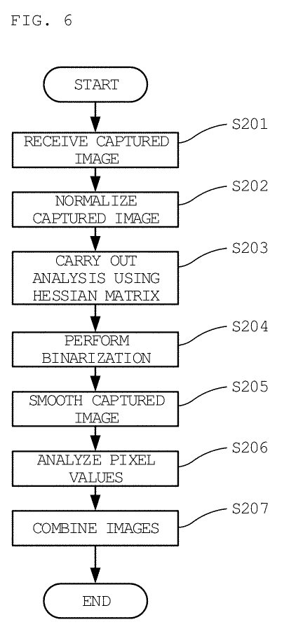

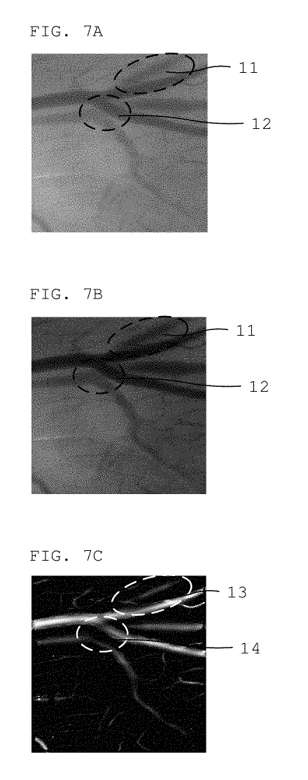

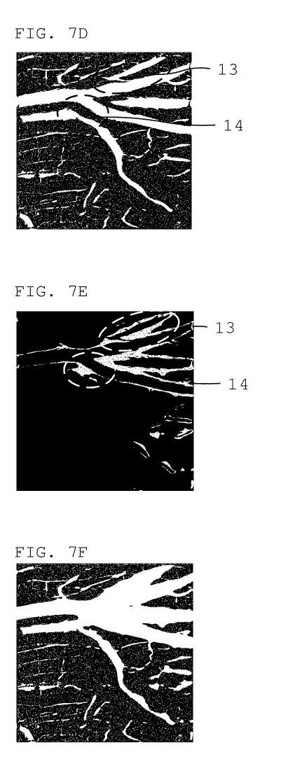

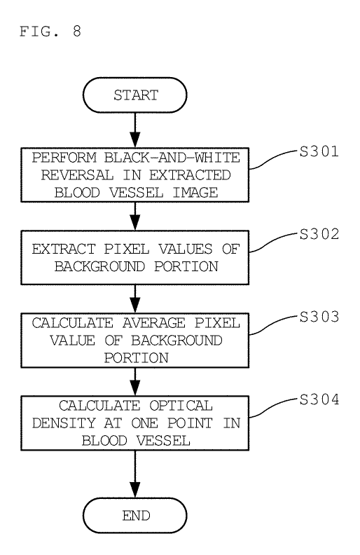

FIG. 7A is a diagram showing an example of the image captured via the probe 200 according to the embodiment of the present disclosure. Specifically, FIG. 7A is an image showing an example of the image (a spectral image) showing the blood vessels of a biological tissue imaged by the probe 200 and stored in the memory 117 in S201. As described above, in this embodiment, the respective spectral images captured in the two luminescent colors of blue light and green light are stored. Accordingly, at least two spectral images like the one shown in FIG. 7A are stored, though not shown in the drawing.

Referring back to FIG. 6, the processor 112 reads each spectral image stored in the memory 117, and performs a normalization process for each pixel by a known method (S202). For example, the processor 112 performs a process of increasing the luminance in the image so that the darkest point in the image becomes “black”, and the brightness of the brightest point in the image is maximized. It should be noted that each of the processed images (normalized images) is temporarily stored into the memory 117.

FIG. 7B is a diagram showing an example of an image processed in the information processing apparatus 100 according to the embodiment of the present disclosure. Specifically, FIG. 7B is a diagram showing an example of a normalized image. As is apparent from the comparison with the spectral image shown in FIG. 7A, it becomes possible to make the dark portion (the portion corresponding to the blood vessels in this embodiment) of the image more conspicuous with respect to the background by performing the normalization processing.

Referring back to FIG. 6, the processor 112 then reads each normalized image from the memory 117, and analyzes the images with a Hessian matrix for each pixel, to extract a tubular structure (which is the structure corresponding to the blood vessels) (S203). For this processing, known methods can be used, including the method reported in “A. F. Frangi et al. Multiscale vessel enhancement filtering, Proceedings of MICCAI, 130-137, 1998”. Each image (extracted tubular structure image) after the tubular structure is extracted through the image analysis using a Hessian matrix is temporarily stored into the memory 117.

FIG. 7C is a diagram showing an example of an image processed in the information processing apparatus 100 according to the embodiment of the present disclosure. Specifically, FIG. 7C is a diagram showing an example of an extracted tubular structure image. The portions analyzed as a tubular structure among the blood vessels shown in “black” or in a color close to black in FIG. 7B are subjected to black and white reversal, and are displayed in white.

Referring back to FIG. 6, the processor 112 then reads each extracted tubular structure image from the memory 117 and performs a binarization process for each pixel (S204). Specifically, the processor 112 performs a process of comparing each pixel value indicated by the gray scales from 0 to 255 with a predetermined threshold value, and converting each pixel value into two tones: black and white. The threshold value can be set as desired. Each image (binarized image) subjected to the binarization process is temporarily stored into the memory 117.

FIG. 7D is a diagram showing an example of an image processed in the information processing apparatus 100 according to the embodiment of the present disclosure. Specifically, FIG. 7D is a diagram showing an example of a binarized image. As is apparent from FIG. 7D, the image drawn in the gray scales in FIG. 7C is displayed as an image converted into the two tones: black and white. This makes it possible to speed up the processes that follow.

As shown in FIGS. 7A and 7D, the biological tissue has regions that are displayed in a blurred manner due to a region 12 in which blood vessels overlaps or a region 11 displaced in the depth direction. In a case where a tubular structure extraction process and a binarization process are performed on an image including such regions, there exist regions that are not extracted as a tubular structure (the regions shown in black in regions 13 and 14 in FIGS. 7C and 7D), though these regions should be extracted as a tubular structure (the regions shown in write in the regions 13 and 14 in FIGS. 7C and 7D).

Referring back to FIG. 6, the processor 112 again reads each spectral image of S201 from the memory 117, and performs a smoothing process (S205). This process may be a known smoothing process, such as a process using a moving average filter or a process using a Gaussian filter. Each image (smoothed image) subjected to the smoothing process is temporarily stored into the memory 117.

The processor 112 then reads each smoothed image from the memory 117, and performs an analysis process using pixel values for each pixel in the regions other than the regions analyzed as a tubular structure (blood vessels) as a result of the binarization process in S204 (which is the regions shown in black in FIG. 7D) (S206). Specifically, for each smoothed image, the processor 112 calculates the average pixel value of the entire image. Using the calculated average pixel value as a threshold value, the processor 112 compares the pixel value of each pixel with the threshold value. In a case where the pixel value is smaller than the threshold value, the portion should be recognized as a blood vessel, and the processor 112 assigns a white tone to the portion accordingly. In a case where the pixel value is greater than the threshold value, the processor 112 assigns a black tone to the portion. Each pixel (pixel-value analyzed image) subjected to the analysis process using pixel values is temporarily stored into the memory 117.

FIG. 7E is a diagram showing an example of an image processed in the information processing apparatus 100 according to the embodiment of the present disclosure. Specifically, FIG. 7E is a diagram showing an example of a pixel-value analyzed image. As described above with reference to FIGS. 7C and 7D, in an extracted tubular structure image, there exist regions that are not recognized as a tubular structure, though these regions correspond to blood vessels (the regions 13 and 14 in FIGS. 7C and 7D, for example). In the pixel-value analyzed image shown in FIG. 7E, a white tone is assigned to each region that should be analyzed as a blood vessel in the regions 13 and 14. Accordingly, a combination of the tubular structure extraction process and the pixel value analysis process enables more accurate analysis of blood vessel regions.

Referring back to FIG. 6, the processor 112 reads out the binarized image and the pixel-value analyzed image stored in the memory 117, and performs a process of combining the two images (S207). Any known combining method may be used as the combining method in this process. The image (composite image) after the combining is stored into the memory 117.

FIG. 7F is a diagram showing an example of an image processed in the information processing apparatus 100 according to the embodiment of the present disclosure. Specifically, FIG. 7F shows an example of the composite image. In the composite image, the region shown in a white tone is the region recognized as the blood vessels. As is apparent from FIG. 7F, the regions that cannot be analyzed as blood vessels in FIGS. 7C and 7D are interpolated through the process illustrated in FIG. 7E, so that more accurate analysis of the blood vessel regions can be carried out.

The above process is performed on each spectral image captured in blue light and green light.

In the above manner, the process for extracting blood vessels from each spectral image captured by the probe 200 is completed.

### 6. Optical Density Calculation Process

FIG. 8 is a diagram showing the flow in a process to be performed in the information processing apparatus 100 according to the embodiment of the present disclosure. Specifically, FIG. 8 is a diagram showing the flow in a process to be performed by the processor 112 of the information processing apparatus 100 executing an instruction command stored in the memory 117.

First, the processor 112 reads the composite image (the image generated in S207) stored in the memory 117, and performs a black-and-white reversal process (S301). The reversal process is performed by a known method. In accordance with the image (reversed image) after the reversal, the processor 112 detects the region that has not been recognized as blood vessels in the process shown in FIG. 6, which is the background region. The processor 112 then reads each smoothed image of S205 of FIG. 6 from the memory 117, and extracts the pixel value of each pixel included in the region identified as the background region (S302). The processor 112 then calculates the average pixel value of the background region from the extracted pixel values (S303). It should be noted that the average pixel value of the background region is the average pixel value of the background region surrounding the coordinate position (x, y) of the blood vessel portion at which the optical density is to be calculated. The surrounding background region may be a surrounding region of a predetermined size centered at the coordinate position (x, y), or may be the portion of the background region in the grid that includes the coordinate position (x, y) in a case where the entire image is divided into grids. The processor 112 then calculates the optical density from the pixel value of each pixel and the calculated average pixel value of the background region of the region in the smoothed image corresponding to the region recognized as the blood vessels in FIG. 7F (S304).

Specifically, the optical density D (x, y) in each pixel is calculated according to the following equation (I).

$$\begin{matrix}[ Mathematical Formula 1 ] & \\ D ( x , y ) = - {log}_{10} [ \frac{I ( x , y )}{{I}_{in} ( x , y )} ] & Equation ( I )\end{matrix}$$

In the equation (I), D(x, y) represents the optical density at the coordinate position (x, y), I(x, y) represents the transmitted light intensity at the coordinate position (x, y), and Iᵢₙ (x, y) represents the incident light intensity at the coordinate position (x, y). Here, the transmitted light intensity is the pixel value of the pixel identified by the coordinate position (x, y) of the blood vessel portion in the smoothed image. The incident light intensity is the average pixel value of the background region calculated in S303.

For each smoothed image, the optical density in each pixel is calculated according to the above equation (I). In this manner, the optical density calculation process is completed.

### 7. Oxygen Saturation Level Calculation Process

In accordance with an instruction command stored in the memory 117, the processor 112 performs a process of estimating the oxygen saturation level of blood, using the information obtained through the respective processes shown in FIGS. 6 and 8. Specifically, for each coordinate position (x, y), the oxygen saturation level is estimated according to the following equation (II).

### [Mathematical Formula 2]

D(λ)=[s̅ ε̅_{HbO}, (λ)+(1−s̅)ε̅_{Hb}(λ)]cd Equation (II)

In the equation (II), D(A) represents the optical density at the coordinate position (x, y) calculated in S304, s represents the blood oxygen saturation level at the coordinate position (x, y), ε_{HbO2} and ε_{Hb} represent the molar absorption coefficients of oxygenated hemoglobin and deoxygenated hemoglobin, respectively, c represents the total concentration of hemoglobin, and d represents the vessel diameter.

Here, the oxygen saturation level s is calculated by solving a system of equations: an equation obtained by assigning the respective numerical values calculated from an image captured with blue light to variables, and an equation obtained by assigning the respective numerical values calculated from an image captured with green light to variables. That is, the oxygen saturation level s is calculated according to the following equation (III).

$$\begin{matrix}[ Mathematical Formula 3 ] & \\ S = \frac{{Ψɛ}_{Hb} ( {λ}_{1} ) - {ɛ}_{Hb} ( {λ}_{2} )}{{Δλ}_{2} - {ΨΔλ}_{1}} & Equation ( III )\end{matrix}$$

In the equation (III), W represents the optical density ratio (D(λ₂)/D(λ₁)) between the image captured with the blue light (λ₁) and the image captured with the green light (λ₂) at the coordinate position (x, y), and Δλₙ represents [εHbO₂(λₙ)−εHb(λₙ)] (n being 1 or 2).

According to the above equation (III), the processor 112 estimates the oxygen saturation level(s) at one or more coordinate positions (x, y) corresponding to the blood vessel(s) included in the image.

### 8. Output Process

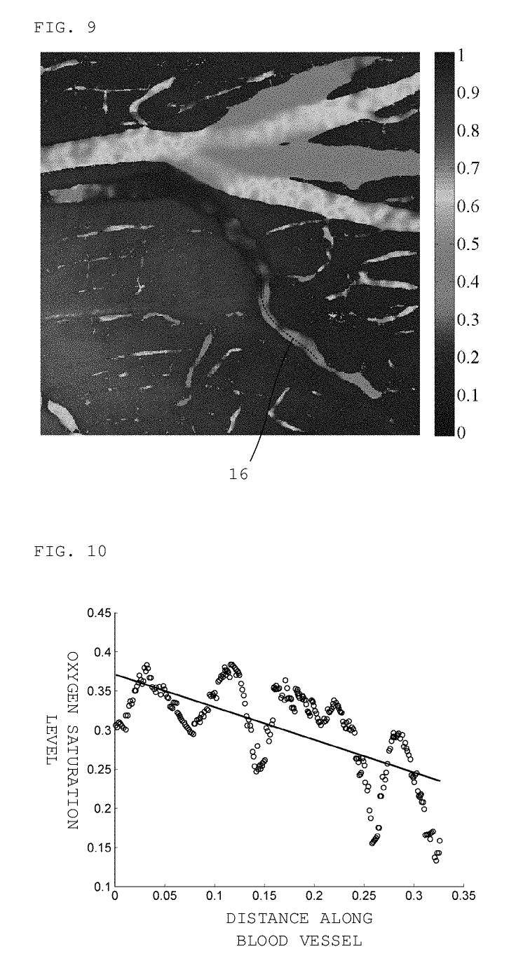

The processor 112 performs a process of outputting the estimated oxygen saturation level as an index indicating the state of the blood in the blood vessel, in accordance with an instruction command stored in the memory 117. FIG. 9 is a diagram showing an example of an image outputted from the information processing apparatus 100 according to the embodiment of the present disclosure. Specifically, the processor 112 classifies tones from blue to red in accordance with the oxygen saturation levels estimated for the respective coordinate positions, and performs control so that the pixels corresponding to the coordinate positions are displayed in the classified tones. At this stage, of the pixels constituting the spectral images stored in the memory 117, the pixels corresponding to the coordinate positions at which oxygen saturation levels have been estimated are replaced with the classified tones, and are then displayed.

As described above, in this embodiment, an oxygen saturation level can be calculated as an index indicating the state of blood in the blood vessel at each coordinate position. Thus, it becomes possible to create a distribution map of oxygen saturation levels of blood, and more accurately analyze the points of high and low oxygen saturation levels.

### 9. Modifications

In the above embodiment, the oxygen saturation level of blood is estimated as an index indicating the state of the blood in a blood vessel. However, it is also possible to estimate the total hemoglobin concentration, instead of or together with the oxygen saturation level. Specifically, the equation (II) has the two unknowns: the oxygen saturation level s and cd, which is the product of the total hemoglobin concentration c and the vessel diameter d. For example, the vessel diameter d can be calculated by a known method, such as setting the half-value width as the vessel diameter from the distribution (profile) of the pixel values in the direction perpendicular to the blood vessel. Therefore, the numerical values necessary in the equation (II) are calculated not only from images obtained from blue light and green light, but also from an image obtained by emitting light in yet another color (blue-green light, for example) having its peak wavelength within the absorption wavelength region of hemoglobin. Thus, it is possible to estimate the total hemoglobin concentration c as well as the oxygen saturation level s.

Although the oxygen saturation level and/or the total hemoglobin concentration are/is used as an index indicating the state of blood in a blood vessel, the index may not be an estimated numerical value, and each estimated numerical value may be divided and classified into predetermined ranges. In other words, the index may be an estimated numerical value, or may be information processed in accordance with the numerical value.

Further, as an index indicating the state of blood in a blood vessel, a predetermined coordinate position in a spectral image is replaced with a predetermined tone and displayed on the display 111. However, it is not always necessary to use spectral images. For example, it is also possible to use normalized images, smoothed images, composite images, or the like stored in the memory 117. Further, when an image is displayed on the display 111, the image is outputted in the form of a map image as shown in FIG. 9. However, the form of a map image is not necessarily used, and a generated index may be displayed at a predetermined position (an upper right portion in the screen, for example) on the display 111, together with an indication line indicating the coordinate position thereof. Although displaying on the display 111 has been described as an output form, images may be outputted from a printer connected to the information processing apparatus 100.

An index indicating the state of blood in a blood vessel is outputted in the form of a two-dimensional map image as shown in FIG. 9. However, oxygen saturation levels estimated along the extracted blood vessel may be plotted in a graph. FIG. 10 is a diagram showing an example of an image outputted from the information processing apparatus 100 according to the embodiment of the present disclosure. Specifically, FIG. 10 shows a graph in which each oxygen saturation level calculated on a line segment 16 in FIG. 9 is plotted for each distance. In this manner, it is also possible to display indices in the form of a graph, instead of the form of a map image, on the display 111.

In the above described embodiment, blue light and green light are cyclically switched and are emitted from the same LEDs 222 of the probe 200. However, LEDs that emit blue light and LEDs that emit green light may be prepared and installed in advance. Also in the above described embodiment, multicolor LEDs are used as light sources, and colors are cyclically switched. However, it is also possible to use white light. In such a case, it is preferable to use a so-called spectroscopic camera, instead of a camera including a conventional image sensor, or to take spectral images of blue light and green light by using a spectral filter.

In the blood vessel extraction process of the above embodiment, the normalization process, the binarization process, the smoothing processing, the analysis process using pixel values, the combining process, and the like are performed. However, it is not necessary to perform these processes. That is, as long as the blood vessel portion can be extracted from each captured spectral image, only the analysis process using a Hessian matrix is performed if a sufficiently high accuracy is guaranteed.

In the above embodiment, the image sensor 212 and the like are disposed in the probe 200. However, the probe 200 is not necessarily provided exclusively for the system 1. That is, it is also possible to provide a light source at the top end portion of an endoscope or a laparoscope, and use the light source as a probe as in this embodiment.

In the above embodiment, a threshold value for determining whether an estimated oxygen saturation level or the total hemoglobin concentration is acceptable is set in advance, and the state of blood in a blood vessel may be reported in accordance with the threshold value. For example, in a case where the blood in a blood vessel is in a poor state, an attention-seeking message, such as “recheck required” or “extra attention required in surgery”, may be displayed on the display 111.

The processes and procedures described in this specification can be realized not only by those explicitly described in the embodiment but also by software, hardware, or a combination thereof. Specifically, the processes and procedures described in this specification can be realized where logics corresponding to the processes are mounted on a medium such as an integrated circuit, a volatile memory, a nonvolatile memory, a magnetic disk, or an optical storage. Also, the processes and procedures described in this specification can be implemented by various computers that store the processes and procedures as computer programs, and include an information processing apparatus and a server apparatus.

Although the processes and procedures described in this specification are performed by a single apparatus, a single set of software, a single component, and a single module, these processes or procedures may be performed by more than one apparatus, more than one set of software, more than one component, and/or more than one module. Also, even though the various kinds of information described in this specification are stored in a single memory or a single storage, such information may be stored in more than one memory provided in a single apparatus or in more than one memory provided in more than one apparatus. Further, the software and hardware components described in this specification may be integrated into a smaller number of components, or may be divided into a larger number of components.

1. An information processing apparatus comprising:
a memory configured to store a predetermined instruction command, and store an image showing a blood vessel of a biological tissue imaged by a probe; and
a processor configured to execute the instruction command stored in the memory, to generate an index indicating a state of blood in the blood vessel at one or a plurality of coordinate positions in the image, and output each generated index associated with each corresponding coordinate position.

2. The information processing apparatus according to claim 1, wherein the index is generated by calculating an oxygen saturation level of the blood.

3. The information processing apparatus according to claim 2, wherein the oxygen saturation level is calculated in accordance with an optical density calculated at the coordinate position of the blood vessel identified from the image.

4. The information processing apparatus according to claim 1, wherein the image is an image captured while the probe is in contact with a surface of the biological tissue.

5. The information processing apparatus according to claim 1, wherein the image is an image captured by a dark field imaging method.

6. The information processing apparatus according to claim 1, further comprising
a display,
wherein the processor executes the instruction command to output the index to the display.

7. The information processing apparatus according to claim 1, wherein the processor executes the instruction command to set and output the index at the coordinate position in the image or in another image generated in accordance with the image.

8. The information processing apparatus according to claim 1, wherein the image is an image captured by emitting a plurality of light beams having different peak wavelength regions.

9. The information processing apparatus according to claim 1, wherein the probe includes an LED light source configured to emit a plurality of light beams having different peak wavelength regions to the biological tissue.

10. The information processing apparatus according to claim 8, wherein each of the peak wavelength regions of the plurality of light beams is included in a light absorption wavelength region of hemoglobin contained in the blood.

11. The information processing apparatus according to claim 10, wherein the plurality of light beams include at least blue light and green light.

12. The information processing apparatus according to claim 1, wherein the coordinate position is a coordinate position corresponding to the blood vessel identified from the image.

13. A computer program product embodying computer-readable instructions stored on a non-transitory computer readable medium for causing a computer to execute a process by a processor, the computer comprising a memory storing an image showing a blood vessel of a biological tissue imaged by a probe,
the computer configured to perform the steps of:
generating an index indicating a state of blood in the blood vessel at one or a plurality of coordinate positions in the image; and
outputting each generated index associated with each corresponding coordinate position.

14. A method implemented by a processor executing a predetermined instruction command stored in a memory,
the method comprising:
storing an image showing a blood vessel of a biological tissue imaged by a probe;
generating an index indicating a state of blood in the blood vessel at one or a plurality of coordinate positions in the image; and
outputting each generated index associated with each corresponding coordinate position.

15. A system comprising:
the information processing apparatus according to claim 1; and
a probe including: a light source that is configured to emit a plurality of light beams having different peak wavelength regions, the light source being communicably connected to the information processing apparatus; and an image sensor that detects light reflected from a surface of a biological tissue among the light beams emitted from the light source.
